(19) United States
(12) Patent Application Publication
(10) Pub. No.: US 2010/0068717 A1
(43) Pub. Date: Mar. 18, 2010

(54) CANCER THERAPY PROGNOSIS

(75) Inventors: Sunil Badve, Indianapolis, IN (US); Harikrishna Nakshatri, Zionsville, IN (US)

Correspondence Address: BARNES & THORNBURG LLP, P.O. BOX 2786, CHICAGO, IL, 60690-2786, US

(73) Assignee: INDIANA UNIVERSITY RESEARCH AND TECHNOLOGY CORPORATION, Indianaplois, IN (US)

(21) Appl. No.: 12/524,845

(22) PCT Filed: Jan. 30, 2008
(86) PCT No.: PCT/US08/52397

§ 371 Date: Aug. 28, 2009

Related U.S. Application Data
(60) Provisional application No. 60/887,290, filed on Jan. 30, 2007.

(51) Int. Cl. C12Q 1/68

(52) U.S. Cl. 435/6

(57) ABSTRACT

Methods and compositions based on FOXA1 expression to predict long-term disease-free survival in patients with breast cancer are disclosed. In ER+ positive patients, expression of FOXA1 is useful in identifying a subgroup of patients with a better prognosis. FOXA1 expression correlates with luminal subtype breast cancer, and serves as a clinical marker for luminal subtype breast cancer. Expression of FOXA1 is useful as a prognostic marker for an effective response tumors and as a predictive marker for a greater likelihood of response to an anti-hormonal therapy. Prognostic ability of FOXA1 in low-risk breast cancers is useful in treatment decision making.

The United States Government may have rights pursuant to funding under grant number NIH CA 089153.

### BACKGROUND

Estrogen (E2) plays an important role in the growth, proliferation and differentiation of mammary epithelium. The estrogen receptors (ERs), ERα and ERβ, mediate the biological action of E2 by functioning as E2-activated transcription factors. ERα is expressed in 10-15% of luminal epithelial cells of normal breast and these cells are generally considered slowly proliferating and well-differentiated cell types. However, more than 50% of breast cancers express ERα at the time of initial diagnosis. These results indicate a distinct role for ERα in the growth of normal, immortalized, and transformed mammary epithelial cells.

Recent gene expression profiling studies have classified breast cancer into five distinct subtypes with unique molecular characteristics and prognostic significance. These include luminal subtypes A and B and others, HER2+/ER−, basal-like, and normal-like subtypes. Luminal subtype A and B correspond to ERα-positive breast cancers with subtype A expressing higher levels of ERα and having a better prognosis than subtype B. Reasons why patients with luminal subtype A tumors have better prognosis than patients with luminal subtype B tumors are not known. It is possible that ERα functions differently in luminal A versus luminal B cancers, which may be due to the influence of additional factors including transcription factors, co-activators and co-repressors that modulate ERα activity.

Transcription factors that are coexpressed with ERα in luminal subtype A include GATA3, and XBP-1. Expression analysis of breast cancer cell lines, as well as patient samples, show clustering of genes regulated by ERα and/or GATA3 into one distinct group and this group correspond to luminal subtype A.

FOXA1/HNF3α, a forkhead family transcription factor interacts with cis-regulatory regions in heterochromatin and enhances the interaction of ERα with chromatin. FOXA1 is required for optimum expression of ˜50% of ERα-regulated genes and estrogen-induced proliferation. A detailed protein analysis relating FOXA1 to ERα and other disease markers such as progesterone receptor (PR), HER2, nodal status, and histologic grade and prognostic significance of such an association have not been reported.

Endocrine therapy is a popular mode of treatment for all stages of breast cancer. A majority of breast cancers belong to the type in which growth is stimulated by the female sex hormones, estrogens and progesterone. Therefore some of the therapies are based on depriving the tumor of the hormone-induced growth stimulus. Some of the current modes of endocrine treatments include blockade of the estrogen receptor with an antiestrogen e.g. tamoxifen; hormonal ablation by surgery (oophorectomy, adrenalectomy or hypophysectomy), radiotherapy or medically by administration of a luteinizing hormone-releasing hormone analogue (LH-RHa) e.g. goserelin; suppression of estrogen synthesis with aromatase inhibitors e.g. anastrozole; pharmacological doses of estrogens and progestagens e.g. megestrol acetate.

The choice of an endocrine agent for breast cancer generally depends on the menopausal status of the patient, the stage of disease, prognostic factors, and the toxicity profile of the agent. Tamoxifen is considered a first-line endocrine therapy for many stages of breast cancer. Some of the new antiestrogens in development include nonsteroidal agents related to tamoxifen and steroidal antiestrogens. Luteinizing hormone-releasing hormone agonists provide an effective form of endocrine therapy for premenopausal women with advanced stage breast cancer, and aromatase inhibitors are sometimes effective in postmenopausal women.

Despite recent advances, the challenge of cancer treatment, including breast cancer therapy remains. Progress is limited with respect to the development of specific treatment regimens to clinically distinct tumor types, and to personalize tumor treatment in order to maximize outcome and efficiency.

Mere classification of breast cancers into a few subgroups characterized by low to absent gene expression of the estrogen receptor (ER) alone may not reflect the cellular and molecular heterogeneity of breast cancer, and may not allow the design of treatment strategies maximizing patient response. Once a patient is diagnosed with cancer, such as breast or ovarian cancer, or an individual wants predisposition analysis, there is a strong need for methods that allow the physician to predict the expected course of disease, including the likelihood of cancer recurrence, long-term survival of the patient, and the like, and accordingly select an appropriate treatment option that is effective.

### SUMMARY

The present disclosure provides a set of genes, the expression of which has prognostic value, specifically with respect to recurrence and disease-free survival of cancer patients, including breast cancer patients. FOXA1 expression predicts disease-free survival in patients with breast cancer. In ER+/PR+/node positive patients, expression of FOXA1 is useful in identifying a subgroup of patients with a better prognosis.

A method of predicting the likelihood of long-term disease-free survival of a breast cancer patient includes the steps of:
(a) determining the expression of a forkhead family transcription factor (FOXA1) in a breast cancer cell obtained from the patient; and
(b) predicting the likelihood of long-term disease-free survival by a statistical analysis of the expression level of FOXA1 in the breast cancer patient.

The breast cancer patient may be estrogen receptor (ER) positive and progesterone receptor (PR) positive. The estrogen receptor may be an alpha subunit (ERα).

In an embodiment, the breast cancer patient is node positive or node negative. In an embodiment, the breast cancer may be of any luminal subtype including A or B or any other sub-type.

In an embodiment, the expression level is determined by analyzing the expression level of RNA transcripts of FOXA1. In an embodiment, the expression level is determined by analyzing the expression level of FOXA1 protein or peptides thereof. In an embodiment, the expression level is determined by analyzing the expression level of FOXA1 in the nucleus of the breast cancer cell.

In an aspect, the likelihood of long-term disease-free survival of the breast cancer patient is predicted by a negative correlation of FOXA1 expression with the expression of mdm-2 (human homolog of mouse double minute 2).

A method of predicting response to endocrine therapy or predicting disease progression in breast cancer includes the steps of:
(a) obtaining a breast cancer test sample from a subject, wherein the subject is suspected of having breast cancer or already has breast cancer;
(b) analyzing the breast cancer test sample for the presence or amount of (1) one or more molecular markers of hormone receptor status selected from the group consisting of estrogen receptor (ER) and progesterone receptor (PR) and (2) a forkhead family transcription factor (FOXA1); and
(c) correlating the presence or amount of the molecular markers with a clinicopathological data from the tissue sample in order to deduce a probability of successful response to endocrine therapy or future risk of disease progression in breast cancer for the subject.

An endocrine therapy is selected from a group of compositions that include tamoxifen, anastrozole, letrozole or exemestane.

Suitable clinicopathological data includes tumor nodal status, tumor grade, tumor size, tumor location, patient age, previous personal and/or familial history of breast cancer, previous personal and/or familial history of response to breast cancer therapy, and BRCA1&2 status.

Correlating gene or protein expression levels disclosed herein are useful for a clinical detection of disease, disease diagnosis, disease prognosis, or treatment outcome or a combination of any two, three or four of these actions.

Suitable test samples may be obtained from or form a part of fixed, paraffin-embedded tissue, breast cancer tissue biopsy, tissue microarray, fresh tumor tissue, fine needle aspirates, peritoneal fluid, ductal lavage and pleural fluid or a derivative thereof.

A method of predicting the likelihood of long-term survival of a cancer patient without the recurrence of cancer includes the steps of:
(a) determining the expression level of the RNA transcript of FOXA1 or its expression products in a cancer cell obtained from the patient; and
(b) predicting the likelihood of long-term survival without the recurrence of cancer if the cancer cell is positive for ERα and PR expression.

A method of prognosing the likelihood of recurrence-free survival in breast cancer patient includes the steps of:
(a) determining the expression of a forkhead family transcription factor (FOXA1) in a breast cancer tissue obtained from the patient, wherein the patient is positive for estrogen receptor (ER) and progesterone receptor (PR) expression; and
(b) prognosing the likelihood of recurrence-free survival by a statistical analysis of the expression level of FOXA1 in the breast cancer tissue.

A method of predicting the likelihood of long-term survival of a cancer patient without the recurrence of cancer includes measuring the expression level of FOXA1 in a breast cancer patient, wherein the patient is positive for estrogen receptor (ER) and progesterone receptor (PR) expression and determining the likelihood based on the expression level of FOXA1.

A method of screening for agents capable of inducing the expression of FOXA1 gene in a breast cancer cell includes the steps of:
(a) obtaining a breast cancer cell line that is positive for estrogen receptor (ER) and progesterone receptor (PR) expression;
(b) contacting the breast cancer cell line to a candidate agent;
(c) analyzing the expression profile of the cancer cell line; and
(d) determining the candidate agent is capable of inducing the expression of FOXA1 gene in the breast cancer cell, if the cancer cell line expresses FOXA1 gene as compared to a control breast cancer cell not contacted with the candidate agent.

A diagnostic kit to predict the long-term disease-free survival or the effectiveness of hormonal therapy of a breast cancer patient includes one or more reagents to detect the presence of FOXA1, ERα and PR expression. The reagent may also include antibodies to specifically detect the presence of FOXA1, ERα and PR. A diagnostic kit may contain specific reagents for in situ detection of either the transcripts or the expressed protein or peptides of FOXA1, ERα and PR.

### BRIEF DESCRIPTION OF THE DRAWINGS

FIG. 1 shows immunohistochemical analysis of FOXA1 in breast cancer. Normal breast contains rare FOXA1 positive cells (a) whereas invasive carcinomas show three distinct patterns of expression: no expression (b), weak expression (c & d) and strong expression (e & f). Numerical scores: A=3, B=0, C=6, D & E=16 and F=30.

FIG. 2 shows Kaplan-Meier survival curve demonstrating the relationship between FOXA1 expression and survival at 20 years. The numbers of patients in each of these groups is shown.

FIG. 3 illustrates Kaplan-Meier curves showing the relationship between FOXA1 expression and survival of patients with nodal status unknown, node-negative and node-positive tumors. The number of patients in each of these groups along with number of events is also shown.

FIG. 4 shows Cox regression analysis of expression levels of FOXA1, ER, and PR with means of covariate values plotted.

FIG. 5 shows Cox regression analysis of survival function.

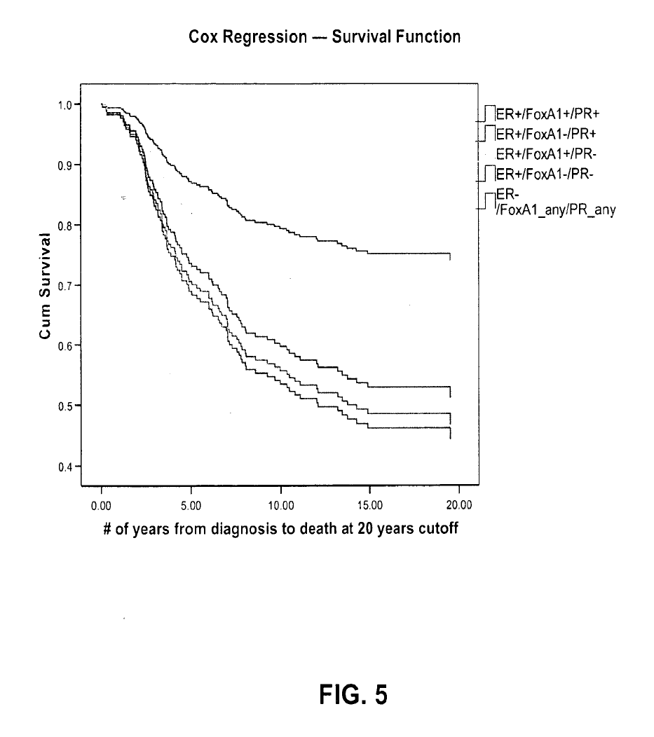

FIG. 6 demonstrates Kaplan-Meier estimates of overall Survival: All patients (p=0.0044) (A); Disease free Survival: All patients (p=0.0543) (B); and Overall Survival: Luminal Subtype patients (p=0.0284) (C).

FIG. 7 demonstrates Kaplan-Meier analysis of cancer-specific survival (all patients) (A); Kaplan-Meier analysis of cancer-specific survival (luminal subtype A) (B); and Kaplan-Meier analysis of cancer-specific survival (ER+ patients)(C).

### DETAILED DESCRIPTION

Analysis of FOXA1 expression, either at the protein level or at the nucleic acid level, provides a novel method to determine the long-term disease-free survival in a cancer patient, including breast cancer patient. Breast cancer patients who are positive for estrogen receptor (ER) and progesterone receptor (PR) expression show a higher likelihood of survival if the expression level of FOXA1 is higher in the breast cancer tissue.

FOXA1 expression in breast cancer patients is associated with ERα-positivity, the luminal A subtype, and better disease-free survival and thus serves as a novel prognostic marker in analyzing breast cancer patients for treatment selection and responsiveness.

FOXA1, a forkhead family transcription factor, is required for optimum expression of about 50% of ERα:E2 responsive genes. FOXA1 is expressed in breast cancer cells and in DNA microarray analyses, segregates with genes that characterize the luminal subtypes. The utility of FOXA1 as a possible independent prognostic factor has not been determined in breast cancers.

FOXA1 expression predicts disease-free survival in patients with cancer, including breast cancer. Aberration in ERalpha:estrogen:FOXA1 network may be responsible in the conversion of ERalpha positive luminal cells from a slowly proliferating and well-differentiated phenotype to a rapidly proliferating aggressive phenotype. Signaling pathways that control these distinct functions of ERα may be responsible in the conversion of ERα-positive luminal epithelial cells from a slowly proliferating and well-differentiated phenotype to a rapidly proliferating aggressive phenotype. Estrogen:ERα dependency of breast cancers for survival/proliferation is related to the expression levels of FOXA1.

Immunohistochemistry (IHC) for FOXA1 expression in breast carcinoma tissues of 438 patients with 20 years follow-up was performed and was compared to FOXA1 expression with various established disease markers and disease-specific survival. FOXA1 expression is associated with ERα-positivity, the luminal A subtype, and better disease-free survival.

Herein, a tissue microarray that includes tumors from 438 patients with 20 years follow-up, was analyzed for FOXA1 expression by immunohistochemistry. FOXA1 expression was examined relative to tumor grade, nodal status, disease-free survival, and expression of ERα, Progesterone Receptor (PR), GATA3, HER2, EGFR, mdm-2 and luminal A subtype (defined as ER+ and/or PR+ and HER2-neg).

FOXA1 expression (score greater than 3) was observed in 300 of 438 breast cancers and correlated with greater likelihood of survival at 20 year (p=0.0004). A significant positive correlation was observed between FOXA1 and ERα (p=0.000001), GATA3 (p=0.000001), PR (p=0.00001), and luminal A subtype (p=0.000001). An inverse correlation was noted with tumor grade, EGFR and mdm-2 expression (p=0.001, 0.000001 and 0.000001, respectively). ER+/PR+/FOXA1+ patients had better long-term survival than ER+/PR+/FOXA1− patients (p=0.005). Survival of ER+/PR+/FOXA1− patients was similar to that of ER+/PR−/FOXA1−(p=0.84) or ER-negative patients (p=0.87).

Patient information and tissue microarray analysis were performed as follows. A tissue microarray (TMA) comprising tumors from 438 patients with 20 years follow-up was analyzed for FOXA1 expression. These samples were obtained from archival cases at Vancouver General Hospital between 1974 and 1995. Patient information, tumor pathology and the expression of a number of biomarkers have been reported in Sutherland et al., (2005), Oncogene, 24:4281-92. In addition to the TMAs, analysis of FOXA1 expression was performed in normal breast tissue obtained from breast reduction specimens. As all personal identifiers had been removed from these samples, information about age or co-existing disease was not available for the normal samples.

IHC for FOXA1 was performed as follows. Expression of FOXA1 was analyzed using goat-anti-human FOXA1 antibody (Santa Cruz Biotechnology, Santa Cruz, Calif., USA) by IHC. After de-waxing and hydration, 4 μm sections from formalin fixed paraffin-embedded tissue were treated with 0.05% citraconic anhydride, pH 7.40 in a decloaking chamber (BioCare®, Walnut Creek, Calif., USA) with chamber settings of SP1: 98° C. for 45 min, and SP2: 0 sec. The slides were then cooled for 20 min at room temperature. Endogenous peroxidase activity was blocked by Peroxo-block (Invitrogen Corporation, Carlsbad, Calif., USA) for 2 min. Non-specific binding of antibodies were blocked with serum-free protein block (Dako Cytomation, Carpentaria, Calif.) for 15 min. The slides were then incubated with polyclonal goat anti-human FOXA1 antibodies (1:250, Santa Cruz Biotechnology, Inc.) for 1 h at room temperature. The sections were incubated with anti-goat HRP polymer conjugate (Invitrogen) according to the manufacturer's instructions. The stain was visualized using DAB plus (Dako Cytomation) and haematoxylin QS (Vector Laboratories, Burlingame, Calif., USA) counterstain. To verify the specificity of staining, non-immune goat serum and PBS controls were used. Percent of staining was categorized as “0” if there was no nuclear expression, 1 for up to 10% positive tumor nuclei, “2” for 11-20% and so on till a maximum score of “10”. Intensity was scored as “1+”, “2+” and “3+” for weak, moderate and strong staining, respectively. Percentage (P) and intensity (I) of nuclear expression were multiplied to generate numerical score (S=P×I).

Univariate/Multivariate analysis and statistical methods were performed as follows. FOXA1 expression was compared with tumor grade, nodal status, disease free survival, and expression of ER, PR+, GATA3, HER2, EGFR, mdm-2 and the luminal A subtype (defined as ER and/or PR+, and HER2-neg), see for example, Carey et al., (2006), JAMA 295:2492-502, in Spearman's two-tailed correlation tests. Kaplan-Meier analysis was performed using log-rank test for comparison of linear trends. Cox proportional hazard ratio model was used for the multivariate survival analysis. Univariate Cox regression was utilized to calculate hazard ratios (HR) for the subgroups depending on the ER/FOXA1/PR coexpression. All tests were two-sided. 5% alpha-level was used to determine significance. SPSS (SPSS Inc., Chicago, Ill.) statistical package was used to perform the analysis.

An X-tile version 3.5.0 to define the optimal cut-off point for the negative and positive scores for FOXA1 (Camp et al., (2004) Clin Cancer Res., 10:7252-9. On a training set of patients generated by the program, the greatest difference in linear survival trends was achieved comparing scores 0-3 vs. 4-30 (chi-square=5.1, p=0.024). This X-tile determined cut-off was confirmed on the validation set (chi-square=7.2, p=0.007). This algorithm uses a training-validation approach to define optimal prognostic cut points from continuous or ordinal tumor biomarker scoring data. Using this approach, S-score values of 0-3 were defined as FOXA1_{low}, and 4-30 as FOXA1_{high}.

FOXA1 as a positive prognostic factor in breast cancer, particularly among ERα-positive cases is demonstrated herein. FOXA1 is a “winged helix” transcription factor, which has recently been dubbed as a “pioneer factor” responsible for the recruitment of ERα to the genome. Depletion of FOXA1 protein in MCF-7 breast cancer cells leads to reduced estrogen-dependent gene expression and proliferation. The C-terminal region of FOXA1 interacts with histones H3 and H4 and this interaction is responsible for opening compacted chromatin, see Cirillo et al. (2000), Mol Cell 9:279-89. By opening chromatin, FOXA1 may permit efficient interaction of ERα with its response elements and subsequent interaction of ERα-associated histone modifying enzymes with histones. About half of estrogen-regulated genes contain binding sites for FOXA1. Optimum expression of these estrogen-regulated genes may occur only in cells that co-express ERα and FOXA1 and these cells form part of the populations of cells that are characterized by estrogen-dependent survival and proliferation signaling pathways.

ER+/FOXA1+ patients showed better prognosis compared to ER+/FOXA1− patients. Better prognosis in these patients is less likely due to ERα/FOXA1-dependent expression of metastasis suppressor genes because among node-positive patients, FOXA1-positive and FOXA1-negative tumors were represented at a similar proportion. ER+/FOXA1+ tumors were more differentiated than other tumor types as evident from their histologic grades and were more dependent on estrogen for survival than other tumors.

A negative relationship was observed between FOXA1 and mdm-2 expression (Table 1). Mdm-2 is considered an oncogene, which targets the tumor suppressor p53 for degradation. Mdm-2 is induced through p53-dependent and p53-independent mechanisms in breast cancer cells. Mdm-2 overexpression in breast cancer in the absence of p53 activity, as measured by the expression levels of the other p53-regulated gene p21, is associated with poor prognosis. It is possible that FOXA1 may directly control mdm-2 expression/activity and, as a consequence, may modulate p53 activity in luminal A subtype.

PR-negativity is a marker for the gain of hormone-independent growth properties by ERα-positive breast cancers through increased crosstalk between ERα and growth factor signaling pathways. Patients who are ER+/FOXA1−/PR− have worse prognoses than patients who are ER+/FOXA1+/PR+. It is possible that ER+/FOXA1−/PR− tumors have acquired hormone-independent growth properties because of shifting of ERα function from traditional ERα:FOXA1:E2 regulated pathway to a ERα:growth factor activated signaling pathways. Thus, simultaneous analysis of ERα, FOXA1 and PR− provides an earlier indication for hormone independence and/or ERα:growth factor signaling pathway crosstalk in ERα-positive breast cancers.

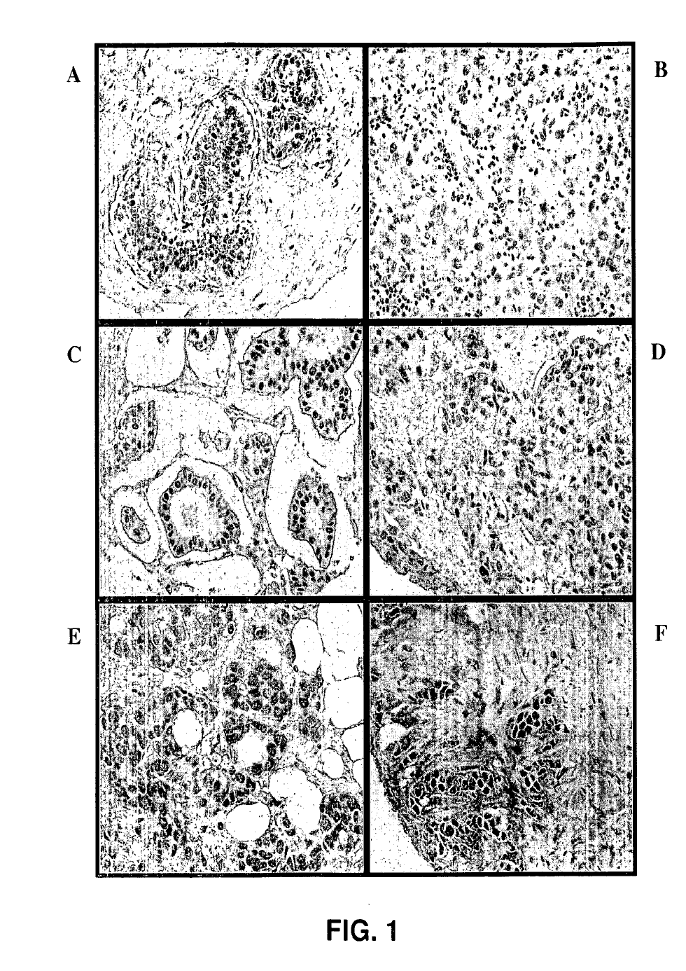

Expression patterns of FOXA1 in normal breast and tumors are substantially similar to that of ERα. In normal breast, ERα expression is observed only in 10-15% of luminal epithelial cells, which is similar to the FOXA1 expression that was observed in the normal breast (FIG. 1). Most of the ERα-positive normal cells are non-dividing and do not express Ki67, a proliferation marker. FOXA1 expression in breast tumors appears not due to cancer-specific overexpression but rather reflects initiation of cancers from luminal cells that express FOXA1 because the intensity of FOXA1 staining in luminal epithelial cells of normal breast and tumors that are highly-positive for FOXA1 is similar.

Immunohistochemistry for FOXA1 expression was performed in breast carcinoma tissues of 438 patients with median follow-up of 15.4 years and FOXA1 expression was compared with various established disease markers and breast cancer-specific survival. The results demonstrate that FOXA1 expression is associated with ERα positivity, the luminal subtype A, and better breast cancer-specific survival.

The functional role of FOXA1 in normal breast is yet to be determined. FOXA1−/− mice are viable and these animals have been analyzed extensively for prostate development. FOXA1−/− animals show a severely altered ductal pattern lacking differentiated or mature luminal epithelial cells. FOXA1, in concert with the androgen receptor, is involved in differentiation of prostate epithelium by regulating the expression of genes such as Nkx3.1, Shh and FOXA2. By analogy, FOXA1 in concert with ERα may be involved in mammary ductal morphogenesis and differentiation. ERα expressing cells have been shown to control proliferation and gene expression pattern in stromal and ERα-negative luminal cells through paracrine mechanisms. This function of ERα may be dependent on FOXA1. Interestingly, FOXA1 expression is regulated by estrogen. Thus, proliferation and differentiation of normal breast epithelium may be under the control of the FOXA1:ERα axis. Because of their mutual dependency for normal function, signaling events that alter the expression levels or function of either of them may be sufficient for initiating transformation of FOXA1+/ERα+ cells or acquisition of estrogen independent growth properties.

Data based on molecular signatures clearly demonstrates the existence of sub-classes within ER-positive tumors which have different clinical courses and different likelihoods of response to therapy. The differences between these classes involve multiple pathways and are not limited to differences in proliferation rates. FOXA1 is an ER-regulated transcription factor that additionally controls downstream transcription of estrogen:ER regulated genes as well as exerts direct control on cell proliferation possibly via regulation of p27^{Kip1}. The utility of FOXA1 expression analysis in identifying and stratifying ER positive patients is determined.

In an embodiment, FOXA1 expression (score >3) was seen in 139/184 breast cancers. It correlated positively with ERα (p<0.0001), PR (p<0.0001), and luminal subtype (p<0.0001); negatively with basal subtype (p<0.0001), proliferation markers and high histological grade (p=0.0327). Univariate analysis showed nodal status, tumour grade, ER, PR, FOXA1, basal markers and p53 as significant predictors of overall survival (OS). Multivariate analysis showed only nodal status (p=0.0006) and ER (p=0.0017) to be the significant predictors of OS. In luminal subtype patient subgroup, FOXA1 expression was associated with better survival (p=0.0284) on univariate analysis.

Validating the cut-off for FOXA1 positivity in a completely independent set of patients who were treated using single standard protocol was performed. The correlations between FOXA1 expression with that of basal and proliferation markers in breast cancer was also determined. FOXA1 is a positive prognostic factor in breast cancer patients (HR=0.36, univariate analysis). FOXA1 expression positively correlated with ER, PR, Cyclin D1 and luminal subtype breast cancers (p<0.0001). This positive correlation with estrogen responsive genes like PR and Cyclin D1 further supports the in-concert action of ER and FOXA1. FOXA1 correlated negatively with high tumor grade, EGFR, p53, MIB1 and basal markers like CK5/6, CK14, and CK17. It did not significantly correlate with tumor size, nodal status and HER-2 expression. Validation of FOXA1 expression by a clinically reproducible method (IHC) adds robustness to the possibility that such expression analysis provides a clinically useful prognostic factor especially in low-risk breast cancer patients.

Luminal subtype breast cancers are good prognosis tumors. FOXA1 expression in luminal subtype breast cancers patients further sub-classifies this group in high and low-risk groups. FOXA1_{neg} and FOXA1ₚₒₛ patients in this subgroup had a 5-year survival of 83.3% and 93.7% respectively, it should be noted here that almost half of the patients in the study had T2 or larger tumors. FOXA1 expression subdivided tumors of luminal profile into two prognostically significant groups. FOXA1 immunohistochemistry is used as a marker for tumors pertaining to the luminal subtype-A breast cancer, which has an exceptionally good prognosis. Studies comparing in parallel FOXA1 IHC evaluation and gene-expression profiling are performed.

FOXA1 expression in luminal subgroup did not correlate with proliferation markers like MIB1 and Cyclin D1, pointing to a mechanistic role. It should however be noted here that this subgroup analysis lacked sufficient statistical power.

Adjuvant treatment decisions, especially decisions like whether to give chemotherapy or not in ER positive-lymph node negative patient subgroup are difficult to make. A prognostic marker that can further identify patients with very low risk of recurrence is useful to avoid toxic therapies in patients who are not likely to derive any additional benefits. FOXA1 expression showed a trend towards better survival in this subgroup. FOXA1 can be used as a prognostic marker in low-risk patients.

The prognostic role of FOXA1 expression by immunohistochemistry in patients treated with current regimens of adjuvant chemotherapy was demonstrated. FOXA1 expression strongly correlated with ER expression and luminal subtype of breast cancer and divides luminal subtype tumors into two distinct prognostic groups: tumors expressing FOXA1 have significantly better survival and are most likely to be luminal subtype-A tumors. FOXA1 is a promising marker for identification of luminal subtype-A tumors in the clinic.

Generally, luminal subtypes e.g., A and B subtypes may be further subdivided into subtypes such as C, and possibly as other subtypes as well. Therefore, the term “luminal subtypes” is intended to encompass all subtype classification of breast cancer that fall under the “luminal” category. Therefore, the term “luminal subtype” is not limited to currently existing nomenclature but includes any further subtype classification. The term “non-A subtype” refers to all luminal subtypes that are not classified as subtype A and thus encompasses subtypes other than subtype A.

Nucleic acid or nucleic acid sequence or polynucleotide or polynucleotide sequence refers to the sequence of a single- or double-stranded DNA or RNA molecule of genomic or synthetic origin, i.e., a polymer of deoxyribonucleotide or ribonucleotide bases, respectively.

Analysis of expression levels (RNA or protein or both) of FOXA1 and/or ERα, PR is useful in prognosing the disease-free survival or recurrence rate in breast cancer patients. Such analysis is also useful in prognosing disease-free survival or recurrence rate in cancers that have differential FOXA1 expression. Long-term disease-free survival indicates that a patient is substantially free of cancer, e.g., metastasis for a significant period, e.g., 3-5 years or longer periods.

Detection of expression of FOXA1 and any other genes include detecting the expression of RNA and the resulting protein products thereof. For example, FOXA1 RNA can be detected using in situ RT-PCR or standard PCR or through any hybridization techniques that involve probes. RNA expression can also be quantified by any known quantification PCR (qPCR) and competitive PCR technology. Microarrays are also useful in quantifying FOXA1 gene expression. RNA can be extracted from a cancer tissue, converted into cDNA and quantified using any known method. RNA can also be directly quantified in situ, within the cancer tissue itself. Probes specific to FOXA1 and ERα and PR may display a high stringency hybridization. Similar techniques are also useful in determining the expression pattern and quantity of other genes such as ERα and PR in the cancer tissues.

FOXA1 protein detection can also be carried in situ or after protein extraction from cancer tissue. Antibodies including monoclonal antibodies against FOXA1 are useful in quantifying FOXA1 protein. Standard immuno histochemistry techniques are capable of detecting the presence and the amount of FOXA1 Antibodies to FOXA1 are either directed to the full length FOXA1 protein or a peptide fragment thereof. Binding specificity of the antibodies to FOXA1 and ER and PR or any other marker disclosed herein conforms to the standards used in immunohistochemistry methodology. The antibodies include monoclonal or polyclonal antibodies.

“Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer. As used herein, “prognostic for cancer” means providing a forecast or prediction of the probable course or outcome of the cancer. In some embodiments, “prognostic for cancer” comprises providing the forecast or prediction of (prognostic for) any one or more of the following: duration of survival of a patient susceptible to or diagnosed with a cancer, duration of recurrence-free survival, duration of progression free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis in a patient susceptible to or diagnosed with a cancer.

A “predictive marker” as used herein refers to a factor that indicates sensitivity or resistance to a specific treatment. Thus, a predictive marker provides a measure of likelihood of response or resistance to a particular therapy.

For example, FoxA1 expression is used as both a prognostic marker (e.g., better response in ER positive tumors) and as a predictive marker (greater likelihood of response to anti-hormonal therapy).

A “sample” (also used as “biological sample” or “tissue or cell sample”) includes a variety of sample types obtained from an individual and can be used in a diagnostic or monitoring assay. The term includes blood and other liquid samples of biological origin, solid tissue samples such as a biopsy specimen or tissue cultures or cells derived therefrom, and the progeny thereof. The definition also includes samples that have been manipulated in any way after their procurement, such as by treatment with reagents, solubilization, or enrichment for certain components, such as proteins or polynucleotides, or embedding in a semi-solid or solid matrix for sectioning purposes. The term “biological sample” encompasses a clinical sample, and also includes cells in culture, cell supernatants, cell lysates, serum, plasma, biological fluid, and tissue samples. The source of the biological sample may be solid tissue as from a fresh, frozen and/or preserved organ or tissue sample or biopsy or aspirate; blood or any blood constituents; bodily fluids such as cerebral spinal fluid, amniotic fluid, peritoneal fluid, or interstitial fluid; cells from any time in gestation or development of the subject. In some embodiments, the biological sample is obtained from a primary or metastatic tumor. The biological sample may contain compounds which are not naturally intermixed with the tissue in nature such as preservatives, anticoagulants, buffers, fixatives, nutrients, antibiotics, or the like.

A “section” of a tissue sample is meant a single part or piece of a tissue sample, e.g. a thin slice of a breast cancer tissue or cells cut from a tissue sample. In some embodiments, the same section of tissue sample is analyzed at both morphological and molecular levels, or is analyzed with respect to both protein and nucleic acid.

By “gene” is meant any polynucleotide sequence or portion thereof with a functional role in encoding or transcribing a protein or regulating other gene expression. The gene may consist of all the nucleic acids responsible for encoding a functional protein or only a portion of the nucleic acids responsible for encoding or expressing a protein. The polynucleotide sequence may contain a genetic abnormality within exons, introns, initiation or termination regions, promoter sequences, other regulatory sequences or unique adjacent regions to the gene.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments.

The term “therapeutically effective amount” refers to an amount of the drug that may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

Examples of cancers include breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, lymphoma, glioma, and multidrug resistant cancer.

As used herein, “treatment” or “therapy” is an approach for obtaining beneficial or desired clinical results. This includes: reduce the number of cancer cells; reduce the tumor size; inhibit (i.e., slow to some extent and/or stop) cancer cell infiltration into peripheral organs; inhibit (i.e., slow to some extent and/or stop) tumor metastasis; inhibit, to some extent, tumor growth; and/or relieve to some extent one or more of the symptoms associated with the disorder, shrinking the size of the tumor, decreasing symptoms resulting from the disease, increasing the quality of life of those suffering from the disease, decreasing the dose of other medications required to treat the disease, delaying the progression of the disease, and/or prolonging survival of patients.

In an embodiment analysis of expression of FOXA1 and/or ERα, PR is useful in prognosing the disease-free survival rate in breast cancer patients. In another embodiment, the analysis of expression of FOXA1 and/or ERα, PR is useful in prognosing an individual's responsiveness to hormonal therapy to breast cancer. In another embodiment, the analysis of expression of FOXA1 and/or ERα, PR is useful in prognosing an individual's responsiveness to conventional drug therapies used along with the hormonal therapy or independent of hormonal therapy, which include monoclonal antibodies, specific inhibitors, chemotherapy, or radiation therapy or a combination thereof for cancer. In another embodiment, FOXA1 expression is used to sub-classify breast cancer, e.g., luminal-type sub-groups. In an embodiment, FOXA1 expression is useful to identify or screen patients at an early stage of anti-estrogen resistance. For example, based on FOXA1 expression, it can be identified as whether or not the patient is less likely or more likely to develop anti-estrogen resistance.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below.

A “chemotherapeutic agent” is a chemical compound useful in the treatment of cancer. Specific chemotherapy for cancers include paclitaxel, docetaxel, cisplatin, methotrexate, cyclophosphamide, 5-fluoro uridine, Leucovorin, Irinotecan (CAMPTOSAR™ or CPT-11 or Camptothecin-11 or Campto), Paclitaxel, Carboplatin, doxorubicin, fluorouracil carboplatin, edatrexate, gemcitabine, or vinorelbine or a combination thereof.

Also included in this definition are anti-hormonal agents (or endocrine therapy) that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen, EVISTA™, raloxifene, droloxifene, 4-hydroxytamoxifen, trioxifene, keoxifene, LY117018, onapristone, and FARESTON™, toremifene; anti-progesterones; estrogen receptor down-regulators (ERDs); agents that function to suppress or shut down the ovaries, for example, leutinizing hormone-releasing hormone (LHRH) agonists such as LUPRON™, and ELIGARD™, leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands. Thus the term “hormonal therapy” or “anti-hormonal therapy” refers to the treatment options to to regulate, reduce, block, or inhibit the effects of hormones that can promote the growth of cancer.

Predicting long-term disease-free survival means that the methods disclosed herein are capable of calculating the likelihood of substantially recurrence-free survival with a 90-95% confidence interval (CI). For example, for ER+/PR+ group, a suitable range would be 95% CI 1.21-3.85, with p=0.009.

The identification and analysis of molecular markers, especially those of FOXA1, ERα, and PR, have numerous therapeutic and diagnostic purposes. Clinical applications include, for example, detection of disease; distinguishing disease states to determine prognosis, selection of therapy, and/or prediction of therapeutic response; disease staging; identification of disease processes; prediction of efficacy of therapy; monitoring of patients' trajectories (e.g., prior to onset of disease); prediction of adverse response; monitoring of therapy associated efficacy and toxicity; prediction of probability of occurrence; recommendation for prophylactic measures; and detection of recurrence.

The molecular markers disclosed herein may be detected using any suitable conventional analytical technique including but not limited to, immunoassays, protein chips, multiplexed immunoassays, complex detection with aptamers, chromatographic separation with spectrophotometric detection, mass spectroscopy, cDNA microarrays, and nucleic acid probe hybridization.

In an embodiment, methods to determine whether a patient sample is FOXA1 positive include using cut-off analysis based on the X-tile analysis (Camp et al., 2004, supra). For example, S-score values of 0-3 are considered as FOXA1_{low} and 4-30 as FOXA1_{high} in determining the correlation of FOXA1 expression for predicting long-term survival rates. Other range FOXA1 includes 1-10 versus 11-30. Survival difference is statistically significant with 11-30 doing better (p=0.0431).

Novel methods for diagnosis and prognosis evaluation for breast cancer, as well as methods for screening for compositions which modulate breast cancer and compositions which bind to modulators of breast cancer are provided. In one aspect, the expression levels of genes such as FOXA1, PR and ER are determined in different patient samples for which either diagnosis or prognosis information is desired. An expression profile (either gene expression or protein expression profiles) of a particular sample is a “fingerprint” of the state of the sample and is also considered a “molecular signature” of the state of the cancer. The intensity of expression of the genes of interest along with the temporal-spatial configurations aid in the evaluation of the expression profile for prognosis. Normal tissue may be distinguished from breast cancer tissue, and within breast cancer tissue, different prognosis states (good or poor long term survival prospects, for example) may be determined. In an embodiment, the evaluation of a particular treatment regime (e.g., endocrine therapy) may be evaluated. Similarly, diagnosis may be done or confirmed by comparing patient samples with known signatures.

Molecular signatures or expression profiles identified herein (e.g., co-expression of FOXA1, ER and PR) that increase the likelihood of long-term disease survival, allow screening of drug candidates capable of mimicking or altering a desirable expression profile and thereby converting a poor prognosis profile to a better prognosis profile. This may be done by obtaining breast cancer cell lines with a poor prognosis profile and testing the effects of candidate drugs on the expression profiles of FOXA1, PR and ERα. These methods can also be done on the protein level, that is, evaluating protein expression levels of the breast cancer proteins through antibody techniques or otherwise or at a nucleic acid level by quantifying the presence of RNA transcripts either directly or through indirect amplification techniques.

FOXA1 expression or any other marker expression in the test biological sample (i.e., the biological sample from the patient having cancer or suspected of having cancer) may be compared to a suitable control sample, as is well known in the art. Exemplary controls include comparable normal samples (e.g., normal non-cancerous tissue or cells of the same type as present in the test biological sample), matched normal samples from the same patient, universal control samples, or a normal reference value (also termed a control reference value). As used herein, the term “control” or “control sample” encompasses a normal reference value. Methods for comparison of expression levels (such as presence or absence of or amount of expression) are known in the art.

As discussed herein, expression in a biological sample can be detected by a number of methods which are well-known in the art, including but not limited to, immunohistochemical and/or Western analysis, biochemical enzymatic activity assays, in situ hybridization, Northern analysis and/or PCR analysis of mRNAs, and genomic Southern analysis (to examine, for example, gene deletion or amplification), as well as any one of the wide variety of assays that can be performed by gene, protein, and/or tissue array analysis.

### EXAMPLES

The following examples are to be considered as exemplary and not restrictive or limiting in character and that all changes and modifications that come within the spirit of the disclosure are desired to be protected.

### Example 1

### FOXA1 Expression in Normal Breast and Breast Cancer

Representative FOXA1 immunostaining of normal breast and breast cancers is shown in FIG. 1. FOXA1 expression was observed in a few luminal epithelial cells of the normal breast. The expression was restricted to the nucleus with little or no cytoplasmic staining Invasive cancers showed variable expression from none, to weak, moderate and strong expression. Staining was nuclear in both normal and cancerous tissues. Thus, FOXA1 is expressed in a specific subpopulation of normal luminal epithelial cells.

### Example 2

### Univariate Analysis of FOXA1 Expression

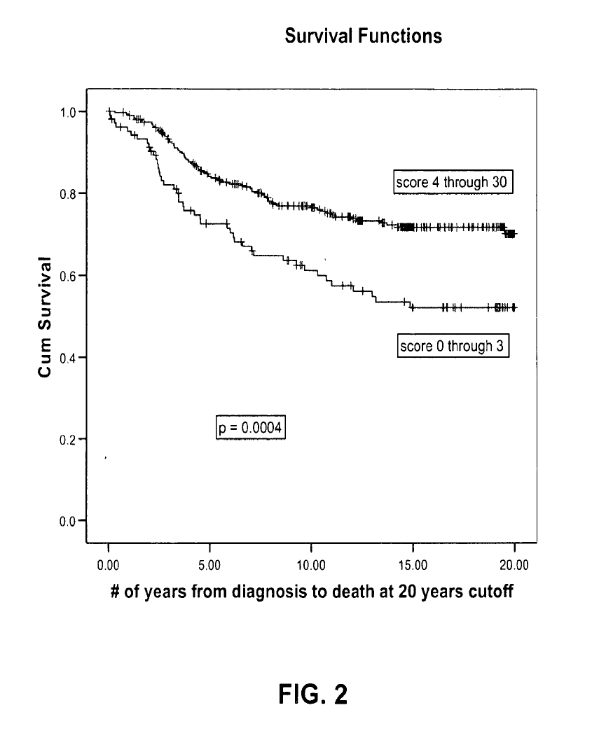

FOXA1 expression (score greater than 3) was seen in 300 of 404 interpretable breast cancers. Based on the expression levels of FOXA1, patients were stratified into two groups; FOXA1_{low} with expression levels of 0-3, and FOXA1_{high} with expression levels of 4-30. Univariate analysis of FOXA1 expression for event-free survival (20 years) showed significantly improved survival of patients in FOXA1_{high} compared to patients in FOXA1_{low} (p=0.0004, FIG. 2). Thus, higher FOXA1 expression correlates with improved survival.

### Example 3

### Bivariant Analysis of FOXA1 Expression with Other Disease Markers

FOXA1 expression was compared with ERα, PR, GATA3, mdm-2, HER2/neu, EGFR, histologic grade, and nodal status as well as luminal A subtype (Table 1). FOXA1 expression correlated significantly with ERα-positivity (Spearman's rho=0.510; p=0.000001) followed by GATA3 (rho=0.511; p=0.000001), PR (rho=0.235; p=0.00001), and luminal A subtype (rho=0.415; p=0.000001). FOXA1 expression correlated negatively with histologic grade (rho=−0.176; p=0.001), mdm-2 (rho=−0.304; p=0.000001), and EGFR (rho=−0.325, p=0.000001). FOXA1 expression did not correlate with nodal status or HER2 expression.

If all 438 patients were considered in a multivariate analysis using a Cox regression model including tumor grade, nodal status, ER, HER2/neu, FOXA1 and GATA3 as variables, FOXA1 did not add any prognostic information in this model (p=0.208 for FOXA1). A significant prognostic factor is the nodal status and subset analysis was performed as described herein.

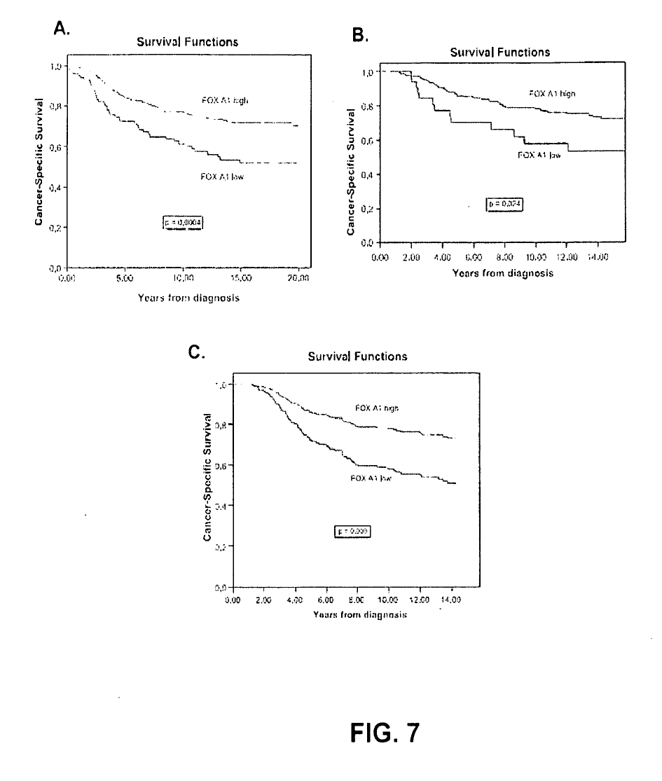

We compared FOXA1 expression with ERα, PR, HER2/neu, histologic grade, nodal status, and luminal subtype A. FOXA1 expression correlated significantly with ERα(P=0.000001), PR (P=0.00001), and luminal subtype A(P=0.000001) (FIG. 7). Eighty-four percent (205 of 244) of hormone receptor-positive patients showed high FOXA1 expression compared with only 32% (16 of 50) of hormone receptor negative patients. Similarly, 84% (168 of 200) of luminal subtype A patients showed high FOXA1 expression compared with only 40.67% (24 of 59) of luminal subtype B. Worsening histologic grade also showed loss of FOXA1 expression (P=0.001). FOXA1 expression did not correlate with nodal status or HER2/neu expression.

### Example 4

### FOXA1 Expression Correlates with Better Prognosis Among Node-Positive Patients

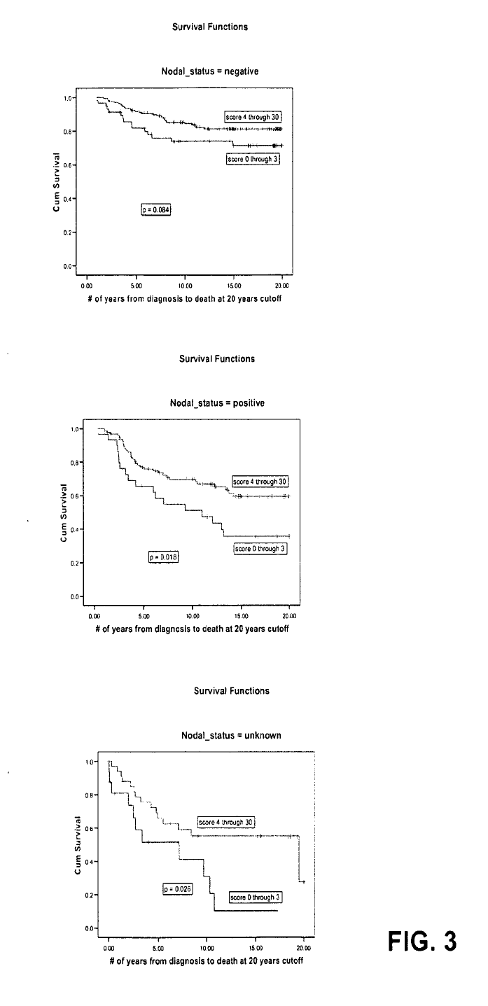

Patients were subclassified into node-positive, node-negative and unknown nodal status and determined the significance of FOXA1 expression within these patient groups. Node-negative patients had a better prognosis than the other two groups, and there was no difference in survival between FOXA1-positive and FOXA1-negative patients within this group (p=0.084, FIG. 3). In contrast, FOXA1-positive patients in both node-positive and unknown nodal status group had a better prognosis than FOXA1-negative patients (p=0.018 and 0.026, respectively). Thus, FOXA1 expression was associated with better prognosis for a specific subgroup of this series of breast cancer patients.

### Example 5

### Prognostic Significance of FOXA1 in Combination with ERα and PR

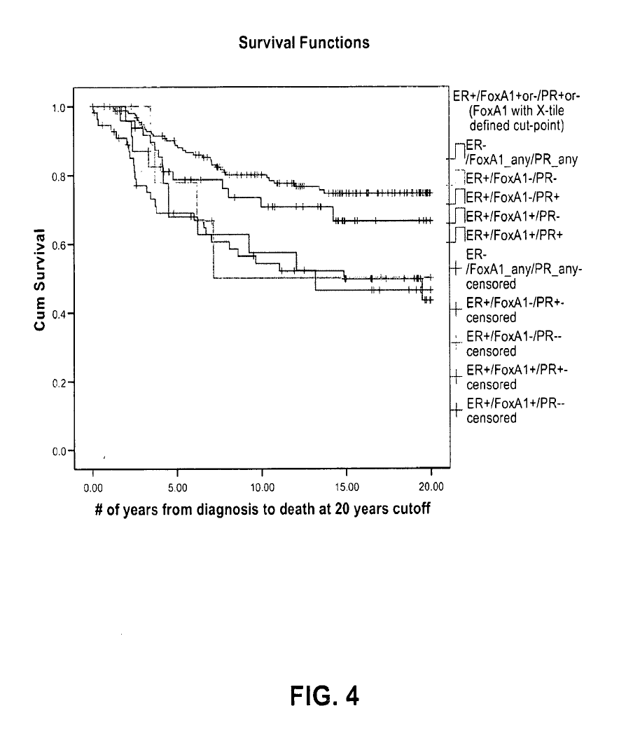

PR has been used as a marker for predicting response to endocrine therapy and event-free survival of patients with ERα-positive breast cancers. Endocrine therapy-resistant ERα-positive tumors often show loss of PR expression, likely due to switching of cell survival from ERα:E2 dependent signaling to ERα-growth factor crosstalk-dependent signaling. To determine whether FOXA1 expression is of any prognostic significance within the context of PR expression, patients were classified into five groups: ER+/PR+/FOXA1+, ER+/PR+/FOXA1−, ER+/PR−/FOXA1+, and ER+/PR−/FOXA1−, and ER−/PR±/FOXA1±. The number of patients in each of these groups and number of events is shown in Table 2. Note that majority of patients are in the ER+/PR+/FOXA1+ group. Poor prognosis group was represented by the ER phenotype (HR=2.71, p=0.0001, FIG. 4), followed by ER+/FOXA1−/PR+(HR=2.53, p=0.007), as showed by Cox regression. PR-negativity did not appear to be a significant prognostic factor as ER+/FOXA1+/PR− group and ER+/FOXA1+/PR+ group showed similar survival profiles with non-significant difference (p=0.26, Kaplan-Meier analysis with pairwise comparisons). Additionally, event-free survival of patients with ER+/FOXA1−/PR− or ER+/FOXA1−/PR+ patients was similar to that of ER-negative patients (p=0.70 and 0.84, respectively). Disease-specific survival of patients with ER+/PR+/FOXA1− was significantly worse than ER+/PR+/FOXA1+ breast cancers (p=0.005). Thus, FOXA1 expression predicts survival of patients within the ER+/PR+breast cancers. However, it did not appear to be useful within the ER+/PR subgroup (p=0.38), although the number of patients in these categories were relatively small.

Two post hoc subgroup analyses were performed: luminal subtype A subgroup and ER-positive lymph node negative subgroup. Luminal subtype A patients expressing high FOXA1 had significantly better survival with relative risk of death due to breast cancer being 0.60 (P=0.024). ER-positive lymph node-negative patients expressing high FOXA1 also showed a trend toward better survival with relative risk of death due to breast cancer being 0.63 (P=0.215 NS). Five and 10-year cancer-specific survival in ER-positive lymph node negative patients expressing high FOXA1 was 92.6 F 2.5% and 85.4 F 3.5%, respectively, compared with 80.0 F 10.3% and 72.7 F 11.7% in FOXA1_{low} patients.

### Example 6

### Prognostic Significance of FOXA1 in a Breast Cancer Tissue Microarray Analysis

A tissue microarray (TMA) was constructed with primary breast cancer samples with replicate 0.6 mm cores of 245 invasive breast carcinomas (186 invasive ductal carcinomas, 27 invasive lobular carcinomas, 24 invasive mixed carcinomas and 8 invasive breast carcinomas of other special types). These samples were obtained from patients primarily treated with therapeutic surgery (69 mastectomies and 155 wide local excisions) at Royal Marsden Hospital, London, UK. Patients in this series were selected on the basis of having received standard anthracycline-based adjuvant chemotherapy. Adjuvant endocrine therapy was prescribed for all patients with ER positive tumors (tamoxifen alone in 96.4% of the patients for the available follow-up period). Follow-up data were available for 244 patients, ranging from 0.5 to 125 months (median—67 months, mean—67 months). Patient information, pathological characteristics of the tumors, detailed TMA preparation and the expression of a number of biomarkers can be found in Reis-Filho et al., (2006) Mod Pathol.; 19(2):307-19. and summarized in Table 3. This study was approved by The Royal Marsden Hospital Research Ethics Committee.

This TMA has already been subjected to immunohistochemical analysis to evaluate expression of ER, PR, HER-2/neu, CK5/6, CK14, CK17, p53, EGFR, MIB1, topoisomerase II α and cyclin D1.

Expression of FOXA1 was analyzed using goat-anti-human FOXA1 antibody (Santa Cruz Biotechnology, Santa Cruz, Calif., USA) by IHC. After de-waxing and hydration, 4 μm sections from formalin fixed paraffin-embedded tissue were treated with 0.05% citraconic anhydride, pH 7.40 in a decloaking chamber (BioCare®, Walnut Creek, Calif., USA) with chamber settings of SP1: 98° C. for 45 min. The slides were then cooled for 20 min at room temperature. Endogenous peroxidase activity was blocked by Peroxo-block (Invitrogen Corporation, Carlsbad, Calif., USA) for 2 min. The slides were then incubated with polyclonal goat FOXA1 antibodies (1:200, Santa Cruz Biotechnology, Inc.) for 1 h at room temperature. The sections were incubated with anti-goat HRP polymer conjugate (Biocompare, San Francisco, Calif., USA). The stain was developed using DAB plus (Dako) and haematoxylin QS (Vector Laboratories, Burlingame, Calif., USA) counterstain. To verify the specificity of staining, non-immune goat serum and PBS negative controls were used. A previously described semi-quantitative scoring system was used; briefly, percentage of staining was categorized as “0” if there was no nuclear expression, “1” for up to 10% positive tumor nuclei, “2” for 11-20% and so on till a maximum score of “10”. Intensity was scored as “1+”, “2+” and “3+” for weak, moderate and strong staining respectively. Percentage (P) and intensity (I) of nuclear expression were multiplied to generate numerical score (S=P×I). Scores of 0-3 were defined as FOXA1_{neg} and 4-30 as FOXA1ₚₒₛ.

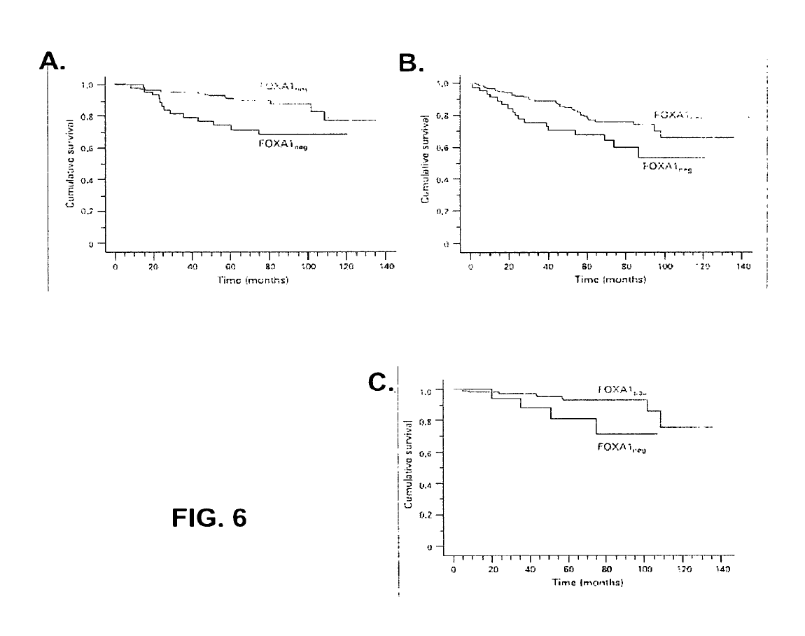

FOXA1 expression correlation with tumor size, histological type, tumor grade, nodal status, lymphovascular invasion (LVI), expression of ER, PR, HER2, proliferation markers like cyclin D1, MIB1, topoisomerase IIα, basal subtype markers like CK5/6, CK14, EGFR, CK17, p53, and breast cancer Subtypes like luminal (defined as ER+ and HER2-neg), basal, and HER-2 was tested. Information regarding each of these parameters was not available for all patients; number of patients for whom information was available is given parameter-wise in Table 4. Kaplan-Meier analysis was performed using log-rank test for comparison of linear trends with overall survival (OS) as primary endpoint (FIG. 6). Cox proportional hazard ratio model was used for the multivariate survival analysis. Univariate Cox regression to calculate hazard ratios (HR) was used for the subgroups. All tests were two-sided. A 5% alpha-level was used to determine significance. Statview 5.0 (SAS Institute, Cary, N.C., USA) statistical package was used for all statistical analyses.

FOXA1 expression was restricted to the nucleus with negligible cytoplasmic staining Tumors showed variable expression: none (24.5%), weak (10.3%), moderate (32.6%) and strong (32.6%). Staining was nuclear in both normal and cancerous tissues. High level of FOXA1 expression (FOXA1pos; score greater than 3) was seen in 139 of 184 (75.5%) interpretable breast cancers.

Correlation of FOXA1 expression with other disease markers was performed. FOXA1 expression was compared with histological grade, nodal status, lymphovascular invasion (LVI), expression of ER, PR, HER2, p53, cyclin D1, MIB1, topoisomerase IIα, basal subtype markers like CK5/6, CK14, EGFR, CK17, and luminal subtype (Table 4). FOXA1 expression correlated positively with ERα (p<0.0001), PR (p<0.0001), and luminal subtype (p<0.0001) and negatively with basal subtype (p<0.0001). FOXA1 expression showed an inverse correlation with high histological grade (p=0.0327) and proliferation as defined by MIB1 (Ki67, p<0.0001). In addition, a direct correlation between the expression of FOXA1 and cyclin D1 (p=0.0012), a gene whose transcription is activated by ER, was found. No significant correlation between FOXA1 expression and tumor size, histological type, nodal status, lymphovascular invasion, topoisomerase IIα or HER2/neu expression was found.

Survival analysis was performed using a variety of statistical tools. Univariate analysis is shown in Table 5 for nodal status, tumor grade, ER, PR, FOXA1 (HR=0.3593 95% CI 0.1723-0.7494), basal markers including p53, MIB1, Cyclin D1, and molecular subtypes as significant predictors of overall survival. FOXA1 was a significant predictor of metastasis free survival (p=0.0185) and had a (p=0.0543) for disease free survival endpoint.

| Homo sapiens forkhead box A1 (FOXA1), Accession No. NM_004496 | |
|---|---|
| (SEQ ID NO: 1) | |
| 1 | atgttaggaa ctgtgaagat ggaagggcat gaaaccagcg actggaacag ctactacgca |
| 61 | gacacgcagg aggcctactc ctccgtcccg gtcagcaaca tgaactcagg cctgggctcc |
| 121 | atgaactcca tgaacaccta catgaccatg aacaccatga ctacgagcgg caacatgacc |
| 181 | ccggcgtcct tcaacatgtc ctatgccaac ccgggcctag gggccggcct gagtcccggc |
| 241 | gcagtagccg gcatgccggg gggctcggcg ggcgccatga acagcatgac tgcggccggc |
| 301 | gtgacggcca tgggtacggc gctgagcccg agcggcatgg gcgccatggg tgcgcagcag |
| 361 | gcggcctcca tgaatggcct gggcccctac gcggccgcca tgaacccgtg catgagcccc |
| 421 | atggcgtacg cgccgtccaa cctgggccgc agccgcgcgg gcggcggcgg cgacgccaag |
| 481 | acgttcaagc gcagctaccc gcacgccaag ccgccctact cgtacatctc gctcatcacc |
| 541 | atggccatcc agcaggcgcc cagcaagatg ctcacgctga gcgagatcta ccagtggatc |
| 601 | atggacctct tcccctatta ccggcagaac cagcagcgct ggcagaactc catccgccac |
| 661 | tcgctgtcct tcaatgactg cttcgtcaag gtggcacgct ccccggacaa gccgggcaag |
| 721 | ggctcctact ggacgctgca cccggactcc ggcaacatgt tcgagaacgg ctgctacttg |
| 781 | cgccgccaga agcgcttcaa gtgcgagaag cagccggggg ccggcggcgg gggcgggagc |
| 841 | ggaagcgggg gcagcggcgc caagggcggc cctgagagcc gcaaggaccc ctctggcgcc |
| 901 | tctaacccca gcgccgactc gcccctccat cggggtgtgc acgggaagac cggccagcta |
| 961 | gagggcgcgc cggcccccgg gcccgccgcc agcccccaga ctctggacca cagtggggcg |
| 1021 | acggcgacag ggggcgcctc ggagttgaag actccagcct cctcaactgc gccccccata |
| 1081 | agctccgggc ccggggcgct ggcctctgtg cccgcctctc acccggcaca cggcttggca |
| 1141 | ccccacgagt cccagctgca cctgaaaggg gacccccact actccttcaa ccacccgttc |
| 1201 | tccatcaaca acctcatgtc ctcctcggag cagcagcata agctggactt caaggcatac |
| 1261 | gaacaggcac tgcaatactc gccttacggc tctacgttgc ccgccagcct gcctctaggc |
| 1321 | agcgcctcgg tgaccaccag gagccccatc gagccctcag ccctggagcc ggcgtactac |
| 1381 | caaggtgtgt attccagacc cgtcctaaac acttcctag (SEQ ID NO: 1) |
| Homo sapiens estrogen receptor 1 (ESR1), Accession No. NM_000125 | |
| (SEQ ID NO: 2) | |
| 1 | atgaccatga ccctccacac caaagcatcc gggatggccc tactgcatca gatccaaggg |
| 61 | aacgagctgg agcccctgaa ccgtccgcag ctcaagatcc ccctggagcg gcccctgggc |
| 121 | gaggtgtacc tggacagcag caagcccgcc gtgtacaact accccgaggg cgccgcctac |
| 181 | gagttcaacg ccgcggccgc cgccaacgcg caggtctacg gtcagaccgg cctcccctac |
| 241 | ggccccgggt ctgaggctgc ggcgttcggc tccaacggcc tggggggttt ccccccactc |
| 301 | aacagcgtgt ctccgagccc gctgatgcta ctgcacccgc cgccgcagct gtcgcctttc |
| 361 | ctgcagcccc acggccagca ggtgccctac tacctggaga acgagcccag cggctacacg |
| 421 | gtgcgcgagg ccggcccgcc ggcattctac aggccaaatt cagataatcg acgccagggt |
| 481 | ggcagagaaa gattggccag taccaatgac aagggaagta tggctatgga atctgccaag |
| 541 | gagactcgct actgtgcagt gtgcaatgac tatgcttcag gctaccatta tggagtctgg |
| 601 | tcctgtgagg gctgcaaggc cttcttcaag agaagtattc aaggacataa cgactatatg |
| 661 | tgtccagcca ccaaccagtg caccattgat aaaaacagga ggaagagctg ccaggcctgc |
| 721 | cggctccgca aatgctacga agtgggaatg atgaaaggtg ggatacgaaa agaccgaaga |
| 781 | ggagggagaa tgttgaaaca caagcgccag agagatgatg gggagggcag gggtgaagtg |
| 841 | gggtctgctg gagacatgag agctgccaac ctttggccaa gcccgctcat gatcaaacgc |
| 901 | tctaagaaga acagcctggc cttgtccctg acggccgacc agatggtcag tgccttgttg |
| 961 | gatgctgagc cccccatact ctattccgag tatgatccta ccagaccctt cagtgaagct |
| 1021 | tcgatgatgg gcttactgac caacctggca gacagggagc tggttcacat gatcaactgg |
| 1081 | gcgaagaggg tgccaggctt tgtggatttg accctccatg atcaggtcca ccttctagaa |
| 1141 | tgtgcctggc tagagatcct gatgattggt ctcgtctggc gctccatgga gcacccaggg |
| 1201 | aagctactgt ttgctcctaa cttgctcttg gacaggaacc agggaaaatg tgtagagggc |
| 1261 | atggtggaga tcttcgacat gctgctggct acatcatctc ggttccgcat gatgaatctg |
| 1321 | cagggagagg agtttgtgtg cctcaaatct attattttgc ttaattctgg agtgtacaca |
| 1381 | tttctgtcca gcaccctgaa gtctctggaa gagaaggacc atatccaccg agtcctggac |
| 1441 | aagatcacag acactttgat ccacctgatg gccaaggcag gcctgaccct gcagcagcag |
| 1501 | caccagcggc tggcccagct cctcctcatc ctctcccaca tcaggcacat gagtaacaaa |
| 1561 | ggcatggagc atctgtacag catgaagtgc aagaacgtgg tgcccctcta tgacctgctg |
| 1621 | ctggagatgc tggacgccca ccgcctacat gcgcccacta gccgtggagg ggcatccgtg |
| 1681 | gaggagacgg accaaagcca cttggccact gcgggctcta cttcatcgca ttccttgcaa |
| 1741 | aagtattaca tcacggggga ggcagagggt ttccctgcca cggtctga (SEQ ID NO: 2) |
| Homo sapiens progesterone receptor (PGR), Accession No. NM_000926 | |
| (SEQ ID NO: 3) | |
| 1 | atgactgagc tgaaggcaaa gggtccccgg gctccccacg tggcgggcgg cccgccctcc |
| 61 | cccgaggtcg gatccccact gctgtgtcgc ccagccgcag gtccgttccc ggggagccag |
| 121 | acctcggaca ccttgcctga agtttcggcc atacctatct ccctggacgg gctactcttc |
| 181 | cctcggccct gccagggaca ggacccctcc gacgaaaaga cgcaggacca gcagtcgctg |
| 241 | tcggacgtgg agggcgcata ttccagagct gaagctacaa ggggtgctgg aggcagcagt |
| 301 | tctagtcccc cagaaaagga cagcggactg ctggacagtg tcttggacac tctgttggcg |
| 361 | ccctcaggtc ccgggcagag ccaacccagc cctcccgcct gcgaggtcac cagctcttgg |
| 421 | tgcctgtttg gccccgaact tcccgaagat ccaccggctg cccccgccac ccagcgggtg |
| 481 | ttgtccccgc tcatgagccg gtccgggtgc aaggttggag acagctccgg gacggcagct |
| 541 | gcccataaag tgctgccccg gggcctgtca ccagcccggc agctgctgct cccggcctct |
| 601 | gagagccctc actggtccgg ggccccagtg aagccgtctc cgcaggccgc tgcggtggag |
| 661 | gttgaggagg aggatggctc tgagtccgag gagtctgcgg gtccgcttct gaagggcaaa |
| 721 | cctcgggctc tgggtggcgc ggcggctgga ggaggagccg cggctgtccc gccgggggcg |
| 781 | gcagcaggag gcgtcgccct ggtccccaag gaagattccc gcttctcagc gcccagggtc |
| 841 | gccctggtgg agcaggacgc gccgatggcg cccgggcgct ccccgctggc caccacggtg |
| 901 | atggatttca tccacgtgcc tatcctgcct ctcaatcacg ccttattggc agcccgcact |
| 961 | cggcagctgc tggaagacga aagttacgac ggcggggccg gggctgccag cgcctttgcc |
| 1021 | ccgccgcgga gttcaccctg tgcctcgtcc accccggtcg ctgtaggcga cttccccgac |
| 1081 | tgcgcgtacc cgcccgacgc cgagcccaag gacgacgcgt accctctcta tagcgacttc |
| 1141 | cagccgcccg ctctaaagat aaaggaggag gaggaaggcg cggaggcctc cgcgcgctcc |
| 1201 | ccgcgttcct accttgtggc cggtgccaac cccgcagcct tcccggattt cccgttgggg |
| 1261 | ccaccgcccc cgctgccgcc gcgagcgacc ccatccagac ccggggaagc ggcggtgacg |
| 1321 | gccgcacccg ccagtgcctc agtctcgtct gcgtcctcct cggggtcgac cctggagtgc |
| 1381 | atcctgtaca aagcggaggg cgcgccgccc cagcagggcc cgttcgcgcc gccgccctgc |
| 1441 | aaggcgccgg gcgcgagcgg ctgcctgctc ccgcgggacg gcctgccctc cacctccgcc |
| 1501 | tctgccgccg ccgccggggc ggcccccgcg ctctaccctg cactcggcct caacgggctc |
| 1561 | ccgcagctcg gctaccaggc cgccgtgctc aaggagggcc tgccgcaggt ctacccgccc |
| 1621 | tatctcaact acctgaggcc ggattcagaa gccagccaga gcccacaata cagcttcgag |
| 1681 | tcattacctc agaagatttg tttaatctgt ggggatgaag catcaggctg tcattatggt |
| 1741 | gtccttacct gtgggagctg taaggtcttc tttaagaggg caatggaagg gcagcacaac |
| 1801 | tacttatgtg ctggaagaaa tgactgcatc gttgataaaa tccgcagaaa aaactgccca |
| 1861 | gcatgtcgcc ttagaaagtg ctgtcaggct ggcatggtcc ttggaggtcg aaaatttaaa |
| 1921 | aagttcaata aagtcagagt tgtgagagca ctggatgctg ttgctctccc acagccagtg |
| 1981 | ggcgttccaa atgaaagcca agccctaagc cagagattca ctttttcacc aggtcaagac |
| 2041 | atacagttga ttccaccact gatcaacctg ttaatgagca ttgaaccaga tgtgatctat |
| 2101 | gcaggacatg acaacacaaa acctgacacc tccagttctt tgctgacaag tcttaatcaa |
| 2161 | ctaggcgaga ggcaacttct ttcagtagtc aagtggtcta aatcattgcc aggttttcga |
| 2221 | aacttacata ttgatgacca gataactctc attcagtatt cttggatgag cttaatggtg |
| 2281 | tttggtctag gatggagatc ctacaaacac gtcagtgggc agatgctgta ttttgcacct |
| 2341 | gatctaatac taaatgaaca gcggatgaaa gaatcatcat tctattcatt atgccttacc |
| 2401 | atgtggcaga tcccacagga gtttgtcaag cttcaagtta gccaagaaga gttcctctgt |
| 2461 | atgaaagtat tgttacttct taatacaatt cctttggaag ggctacgaag tcaaacccag |
| 2521 | tttgaggaga tgaggtcaag ctacattaga gagctcatca aggcaattgg tttgaggcaa |
| 2581 | aaaggagttg tgtcgagctc acagcgtttc tatcaactta caaaacttct tgataacttg |
| 2641 | catgatcttg tcaaacaact tcatctgtac tgcttgaata catttatcca gtcccgggca |
| 2701 | ctgagtgttg aatttccaga aatgatgtct gaagttattg ctgcacaatt acccaagata |
| 2761 | ttggcaggga tggtgaaacc ccttctcttt cataaaaagt ga |

TABLE 1
Bivariant analysis of FOXA1 with other disease markers: *Luminal
type A is defined ER and/or PR+ and Her2-negative.

| | Correlation | Significance | Number of |
|---|---|---|---|
| | coefficient | (2-tailed) | patients |
| ER | 0.510 | 0.000001 | 303 |
| GATA3 | 0.511 | 0.000001 | 356 |
| Luminal type A* | 0.415 | 0.000001 | 259 |
| PR | 0.235 | 0.00001 | 337 |
| Histology grade | −0.176 | 0.001 | 347 |
| Mdm-2 | −0.304 | 0.000001 | 342 |
| Nodal status | 0.025 | 0.643 | 354 |
| Her2 | 0.015 | 0.787 | 317 |

TABLE 2
Number of patients and events in groups stratified
based on FOXA1, ER and PR expression.

| | | Number | Number | |
|---|---|---|---|---|
| | Total | of events | censored | Percent |
| ER+/FOXA1+/PR+ | 156 | 35 | 121 | 77.6 |
| ER+/FOXA1+/PR− | 49 | 14 | 35 | 71.4 |
| ER+/FOXA1−/PR+ | 23 | 11 | 12 | 52.2 |
| ER+/FOXA1−/PR− | 10 | 4 | 6 | 60.0 |
| ER-/FOXA1±/PR± | 56 | 26 | 30 | 53.6 |
| Overall | 294 | 90 | 204 | 69.4 |

TABLE 3
Description of the patients and characteristics of their tumors:

| Parameter | | N | Percentage |
|---|---|---|---|
| Histology | IDC | 186 | 75.9 |
| | ILC | 27 | 11 |
| | Mixed | 24 | 9.8 |
| | Other | 8 | 3.3 |
| | Not assessable | 0 | 0 |
| Size | T1 (<2 cm) | 127 | 51.8 |
| | T2 (2-5 cm) | 100 | 40.8 |
| | T3 (>5 cm) | 16 | 6.5 |
| | Not assessable | 2 | 0.8 |
| Lymph nodes | Positive | 154 | 62.9 |
| | Negative | 83 | 33.9 |
| | Not assessable | 8 | 3.3 |
| Grade | I | 23 | 9.4 |
| | II | 69 | 28.2 |
| | III | 148 | 60.4 |
| | Not assessable | 5 | 2 |
| ER | Positive | 192 | 78.4 |
| | Negative | 48 | 19.6 |
| | Not assessable | 5 | 2 |
| PR | Positive | 175 | 71.4 |
| | Negative | 65 | 26.5 |
| | Not assessable | 5 | 2 |
| LVI | Present | 161 | 65.7 |
| | Absent | 82 | 33.5 |
| | Not assessable | 2 | 0.8 |
| HER2(IHC) | Positive | 32 | 13.1 |
| | Negative | 205 | 83.7 |
| | Not assessable | 8 | 3.3 |

Abbreviations: IDC: infiltrating ductal carcinoma; ILC: infiltrating lobular carcinoma

TABLE 4
Correlations between FOXA1 expression, clinicopathological parameters
and immunohistochemical markers in 245 invasive breast carcinomas.

| | | | FOXA1_{neg} | FOXA1ₚₒₛ | | |
|---|---|---|---|---|---|---|
| Parameter | N | | (%) | (%) | Correlation | p value |
| Size | 182 | T1 | 19 (20) | 79 (80) | — | P = 0.1560* |
| | | T2 | 21 (30) | 50 (70) | | |
| | | T3 | 5 (38) | 8 (62) | | |
| Grade | 180 | I | 2 (12) | 14 (88) | Negative | P = 0.0327* |
| | | II | 8 (15) | 47 (85) | | |
| | | III | 34 (31) | 75 (69) | | |
| Type | 181 | IDC | 32 (25) | 97 (75) | — | P = 0.6177* |
| | | ILC | 5 (17) | 25 (83) | | |
| | | Other | 6 (27) | 16 (73) | | |
| LVI | 182 | − | 19 (28) | 48 (72) | — | P = 0.3702** |
| | | + | 25 (22) | 90 (78) | | |
| LN | 177 | − | 17 (27) | 47 (73) | — | P = 0.7197** |
| metastasis | | + | 27 (24) | 86 (76) | | |
| ER | 183 | − | 24 (73) | 9 (27) | Positive | P < 0.0001** |
| | | + | 20 (13) | 130 (87) | | |
| PR | 183 | − | 23 (50) | 23 (50) | Positive | P < 0.0001** |
| | | + | 21 (15) | 116 (85) | | |
| HER-2 | 183 | − | 39 (25) | 119 (75) | — | P = 0.8020** |
| | | + | 5 (20) | 20 (80) | | |
| EGFR | 184 | − | 32 (19) | 135 (81) | Negative | P < 0.0001** |
| | | + | 13 (76) | 4 (24) | | |
| CK14 | 182 | − | 31 (19) | 135 (81) | Negative | P < 0.0001** |
| | | + | 13 (81) | 3 (19) | | |
| CK5/6 | 176 | − | 32 (20) | 127 (80) | Negative | P = 0.0002** |
| | | + | 11 (65) | 6 (35) | | |
| CK17 | 182 | − | 30 (18) | 133 (82) | Negative | P < 0.0001** |
| | | + | 14 (74) | 5 (26) | | |
| p53 | 182 | − | 21 (16) | 107 (84) | Negative | P = 0.0001** |
| | | + | 24 (44) | 30 (56) | | |
| MIB1 | 180 | <10% | 10 (13) | 67 (87) | Negative | P < 0.0001* |
| | | 10-30% | 19 (24) | 60 (76) | | |
| | | >30% | 16 (67) | 8 (33) | | |
| Cyclin D1 | 182 | Low | 10 (50) | 10 (50) | Positive | P = 0.0012* |
| | | Intermediate | 12 (36) | 21 (64) | | |
| | | High | 22 (17) | 107 (83) | | |
| Topo2A | 180 | Low | 14 (17) | 67 (83) | — | P = 0.0550** |
| | | High | 30 (30) | 69 (70) | | |
| Nielsen | 176 | Basal | 18 (82) | 4 (18) | Negative | P = 0.0001* |
| groups | | Luminal | 18 (14) | 112 (86) | Positive | |
| | | HER-2 | 5 (21) | 19 (79) | Positive | |

CK: cytokeratin; ER: estrogen receptor; LN metastasis: lymph node metastasis; LVI: lymphovascular invasion; Topo2A topoisomerase II α; Subgroups defined according to the definition described by Nielsen et al. [ref 6]
*Chi-square test;
**Fisher's exact test

TABLE 5
Univariate analysis of various parameters (Overall survival).

| Parameter | Comparison | HR (95% CI) | Significance |
|---|---|---|---|
| Nodal status | N0 vs. N+ | 4.65 (1.83-11.83) | 0.0013 |
| Tumor Size | T1 vs. T2 | 1.23 (0.65-2.33) | NS |
| | T1 vs. T3 | 1.46 (0.85-2.52) | |
| FOXA1 | FOXA1ₚₒₛ vs. FOXA1_{neg} | 0.36 (0.17-0.75) | 0.0044 |
| ER | ER+ vs. ER− | 0.31 (0.17-0.58) | 0.0001 |
| Tumor grade | Grade I/II vs. Grade III | 2.26 (0.08-4.72) | 0.0307 |
| HER-2/neu | HER-2+ vs. HER-2− | 1.60 (0.76-3.36) | NS |
| PR | PR+ vs. PR- | 0.34 (0.18-0.63) | 0.0005 |
| Any basal | Positive vs. negative | 2.83 (1.49-5.39) | 0.0010 |
| marker | | | |
| p53 | p53+ vs. p53− | 2.81 (1.49-5.33) | 0.0010 |
| MIB1 | Low vs. Intermediate | 1.40 (0.65-3.02) | 0.0023 |
| | Low vs. High | 1.96 (1.31-2.93) | |
| Cyclin D1 | Intermediate vs. Low | 0.59 (0.23-1.48) | 0.0049 |
| | High vs. Low | 0.57 (0.39-0.85) | |
| Molecular | HER2 vs. luminal | 2.25 (1.01-4.97) | 0.0007 |
| subgroups | Basal vs. luminal | 1.83 (1.26-2.66) | |

TABLE 6
Correlations between FOXA1 expression, clinicopathological
parameters and immunohistochemical markers in
luminal* breast carcinomas.

| | | | FOXA1_{neg} | FOXA1ₚₒₛ | |
|---|---|---|---|---|---|
| Parameter | N | | (%) | (%) | p value |
| Size | 128 | T1 | 8 (11) | 64 (89) | P = 0.5298 |
| | | T2 | 9 (18) | 40 (82) | |
| | | T3 | 1 (14) | 6 (86) | |
| Grade | 127 | I | 2 (13) | 13 (87) | P = 0.9857 |
| | | II | 7 (14) | 43 (86) | |
| | | III | 8 (13) | 54 (87) | |
| Type | 129 | IDC | 12 (14) | 75 (86) | P = 0.8681 |
| | | ILC | 3 (12) | 22 (88) | |
| | | Other | 2 (12) | 15 (88) | |
| LVI | 128 | − | 5 (11) | 39 (89) | P = 0.7868 |
| | | + | 12 (14) | 72 (86) | |
| LN | 125 | − | 3 (17) | 15 (83) | P = 0.1144 |
| metastasis | | + | 15 (18) | 68 (82) | |
| PR | 130 | − | 2 (13) | 13 (87) | P > 0.9999 |
| | | + | 16 (14) | 99 (86) | |
| p53 | 129 | − | 11 (11) | 91 (89) | P = 0.0598 |
| | | + | 7 (26) | 20 (74) | |
| Topo2A | 129 | Low | 9 (15) | 53 (85) | P = >0.9999 |
| | | High | 9 (13) | 58 (87) | |
| MIB1 | 128 | <10% | 8 (12) | 58 (88) | P = 0.7035 |
| | | 10-30% | 9 (16) | 49 (84) | |
| | | >30% | 1 (25) | 3 (75) | |
| Cyclin D1 | 130 | Low | 1 (14) | 6 (86) | P = 0.2096 |
| | | Intermediate | 6 (25) | 18 (75) | |
| | | High | 11 (11) | 88 (89) | |

LN metastasis: lymph node metastasis; LVI: lymphovascular invasion; Topo2A topoisomerase II α
*Luminal cancers were defined according to the definition described by Nielsen et al. Clin Cancer Res. 2004; 10(16): 5367-74.

1. A method of predicting the likelihood of long-term disease-free survival of a breast cancer patient, the method comprising:
(a) determining the expression of a forkhead family transcription factor (FOXA1) in a sample obtained from the patient; and
(b) predicting the likelihood of long-term disease-free survival by a statistical analysis of the expression of FOXA1 in the breast cancer patient.

2. The method of claim 1, wherein the breast cancer patient is estrogen receptor (ER) positive and progesterone receptor (PR) positive.

3. The method of claim 2, wherein the estrogen receptor is an alpha subunit (ERα).

4. The method of claim 1, wherein the breast cancer patient is node positive.

5. The method of claim 1, wherein the expression is determined by analyzing the expression of RNA transcripts of FOXA1.

6. The method of claim 1, wherein the expression is determined by analyzing the expression of FOXA1 protein or peptides thereof.

7. The method of claim 1, wherein the expression is determined by analyzing the expression of FOXA1 in the nucleus of a breast cancer cell in the sample.

8. The method of claim 7, wherein the breast cancer is of luminal subtype is A or non-A.

9. The method of claim 1, wherein the likelihood of long-term disease-free survival of the breast cancer patient is predicted by a negative correlation of FOXA1 expression with the expression of mdm-2 (human homolog of mouse double minute 2).

10. The method of claim 1, wherein the disease-free survival is metastasis free.

11. A method of predicting response to endocrine therapy or predicting disease progression in breast cancer and administering a cancer therapy, the method comprising:
(a) obtaining a breast cancer test sample from a subject;
(b) analyzing the breast cancer test sample for the presence or amount of (1) one or more molecular markers of hormone receptor status selected from the group consisting of estrogen receptor (ER) and progesterone receptor (PR), and (2) a forkhead family transcription factor (FOXA1);
(c) correlating the presence or amount of the molecular markers with a clinicopathological data from the tissue sample in order to deduce a probability of successful response to endocrine therapy or future risk of disease progression of breast cancer for the subject; and
(d) administering the endocrine therapy for the subject.

12. The method of claim 11, wherein the endocrine therapy is selected from the group consisting of tamoxifen, anastrozole, letrozole or exemestane.

13. The method of claim 11, wherein the clinicopathological data is selected from the group consisting of tumor nodal status, tumor grade, tumor size, tumor location, patient age, previous personal and/or familial history of breast cancer, previous personal and/or familial history of response to breast cancer therapy, and BRCA1&2 status.

14. The method of claim 11 wherein the correlating is useful for a clinical detection of disease, disease diagnosis, disease prognosis, or treatment outcome or a combination of any two, three or four of these actions.

15. The method of claim 11, wherein the obtaining of the test sample from the subject is of a test sample selected from the group consisting of fixed, paraffin-embedded tissue, breast cancer tissue biopsy, tissue microarray, fresh tumor tissue, fine needle aspirates, peritoneal fluid, ductal lavage and pleural fluid or a derivative thereof.

16. A method of predicting the likelihood of long-term survival of a breast cancer patient without the recurrence of breast cancer and administering a therapy thereof, comprising:
(a) determining the presence or the expression of the RNA transcript of FOXA1 or its expression products in a cancer tissue sample obtained from the patient;
(b) predicting the likelihood of long-term survival without the recurrence of breast cancer if the cancer cell is positive for FOXA1, ERα and PR expression; and
(c) administering an endocrine therapy to the patient based on the likelihood of long-term survival prediction.

17.-21. (canceled)


SEQUENCE LISTING (3 sequences)

SEQ ID NO: 1 - DNA, 1419 bp, Homo Sapien
atgttaggaactgtgaagatggaagggcatgaaaccagcgactggaacagctactacgcagacacgcaggaggcctactcctccgtcccggtcagcaacatgaactcaggcctgggctccatgaactccatgaacacctacatgaccatgaacaccatgactacgagcggcaacatgaccccggcgtccttcaacatgtcctatgccaacccgggcctaggggccggcctgagtcccggcgcagtagccggcatgccggggggctcggcgggcgccatgaacagcatgactgcggccggcgtgacggccatgggtacggcgctgagcccgagcggcatgggcgccatgggtgcgcagcaggcggcctccatgaatggcctgggcccctacgcggccgccatgaacccgtgcatgagccccatggcgtacgcgccgtccaacctgggccgcagccgcgcgggcggcggcggcgacgccaagacgttcaagcgcagctacccgcacgccaagccgccctactcgtacatctcgctcatcaccatggccatccagcaggcgcccagcaagatgctcacgctgagcgagatctaccagtggatcatggacctcttcccctattaccggcagaaccagcagcgctggcagaactccatccgccactcgctgtccttcaatgactgcttcgtcaaggtggcacgctccccggacaagccgggcaagggctcctactggacgctgcacccggactccggcaacatgttcgagaacggctgctacttgcgccgccagaagcgcttcaagtgcgagaagcagccgggggccggcggcgggggcgggagcggaagcgggggcagcggcgccaagggcggccctgagagccgcaaggacccctctggcgcctctaaccccagcgccgactcgcccctccatcggggtgtgcacgggaagaccggccagctagagggcgcgccggcccccgggcccgccgccagcccccagactctggaccacagtggggcgacggcgacagggggcgcctcggagttgaagactccagcctcctcaactgcgccccccataagctccgggcccggggcgctggcctctgtgcccgcctctcacccggcacacggcttggcaccccacgagtcccagctgcacctgaaaggggacccccactactccttcaaccacccgttctccatcaacaacctcatgtcctcctcggagcagcagcataagctggacttcaaggcatacgaacaggcactgcaatactcgccttacggctctacgttgcccgccagcctgcctctaggcagcgcctcggtgaccaccaggagccccatcgagccctcagccctggagccggcgtactaccaaggtgtgtattccagacccgtcctaaacacttcctag

SEQ ID NO: 2 - DNA, 1788 bp, Homo Sapien
atgaccatgaccctccacaccaaagcatccgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 3 - DNA, 2802 bp, Homo Sapien
atgactgagctgaaggcaaagggtccccgggctccccacgtggcgggcggcccgccctcccccgaggtcggatccccactgctgtgtcgcccagccgcaggtccgttcccggggagccagacctcggacaccttgcctgaagtttcggccatacctatctccctggacgggctactcttccctcggccctgccagggacaggacccctccgacgaaaagacgcaggaccagcagtcgctgtcggacgtggagggcgcatattccagagctgaagctacaaggggtgctggaggcagcagttctagtcccccagaaaaggacagcggactgctggacagtgtcttggacactctgttggcgccctcaggtcccgggcagagccaacccagccctcccgcctgcgaggtcaccagctcttggtgcctgtttggccccgaacttcccgaagatccaccggctgcccccgccacccagcgggtgttgtccccgctcatgagccggtccgggtgcaaggttggagacagctccgggacggcagctgcccataaagtgctgccccggggcctgtcaccagcccggcagctgctgctcccggcctctgagagccctcactggtccggggccccagtgaagccgtctccgcaggccgctgcggtggaggttgaggaggaggatggctctgagtccgaggagtctgcgggtccgcttctgaagggcaaacctcgggctctgggtggcgcggcggctggaggaggagccgcggctgtcccgccgggggcggcagcaggaggcgtcgccctggtccccaaggaagattcccgcttctcagcgcccagggtcgccctggtggagcaggacgcgccgatggcgcccgggcgctccccgctggccaccacggtgatggatttcatccacgtgcctatcctgcctctcaatcacgccttattggcagcccgcactcggcagctgctggaagacgaaagttacgacggcggggccggggctgccagcgcctttgccccgccgcggagttcaccctgtgcctcgtccaccccggtcgctgtaggcgacttccccgactgcgcgtacccgcccgacgccgagcccaaggacgacgcgtaccctctctatagcgacttccagccgcccgctctaaagataaaggaggaggaggaaggcgcggaggcctccgcgcgctccccgcgttcctaccttgtggccggtgccaaccccgcagccttcccggatttcccgttggggccaccgcccccgctgccgccgcgagcgaccccatccagacccggggaagcggcggtgacggccgcacccgccagtgcctcagtctcgtctgcgtcctcctcggggtcgaccctggagtgcatcctgtacaaagcggagggcgcgccgccccagcagggcccgttcgcgccgccgccctgcaaggcgccgggcgcgagcggctgcctgctcccgcgggacggcctgccctccacctccgcctctgccgccgccgccggggcggcccccgcgctctaccctgcactcggcctcaacgggctcccgcagctcggctaccaggccgccgtgctcaaggagggcctgccgcaggtctacccgccctatctcaactacctgaggccggattcagaagccagccagagcccacaatacagcttcgagtcattacctcagaagatttgtttaatctgtggggatgaagcatcaggctgtcattatggtgtccttacctgtgggagctgtaaggtcttctttaagagggcaatggaagggcagcacaactacttatgtgctggaagaaatgactgcatcgttgataaaatccgcagaaaaaactgcccagcatgtcgccttagaaagtgctgtcaggctggcatggtccttggaggtcgaaaatttaaaaagttcaataaagtcagagttgtgagagcactggatgctgttgctctcccacagccagtgggcgttccaaatgaaagccaagccctaagccagagattcactttttcaccaggtcaagacatacagttgattccaccactgatcaacctgttaatgagcattgaaccagatgtgatctatgcaggacatgacaacacaaaacctgacacctccagttctttgctgacaagtcttaatcaactaggcgagaggcaacttctttcagtagtcaagtggtctaaatcattgccaggttttcgaaacttacatattgatgaccagataactctcattcagtattcttggatgagcttaatggtgtttggtctaggatggagatcctacaaacacgtcagtgggcagatgctgtattttgcacctgatctaatactaaatgaacagcggatgaaagaatcatcattctattcattatgccttaccatgtggcagatcccacaggagtttgtcaagcttcaagttagccaagaagagttcctctgtatgaaagtattgttacttcttaatacaattcctttggaagggctacgaagtcaaacccagtttgaggagatgaggtcaagctacattagagagctcatcaaggcaattggtttgaggcaaaaaggagttgtgtcgagctcacagcgtttctatcaacttacaaaacttcttgataacttgcatgatcttgtcaaacaacttcatctgtactgcttgaatacatttatccagtcccgggcactgagtgttgaatttccagaaatgatgtctgaagttattgctgcacaattacccaagatattggcagggatggtgaaaccccttctctttcataaaaagtga